(19) United States
(12) Patent Application Publication
(10) Pub. No.: US 2024/0024609 A1
(43) Pub. Date: Jan. 25, 2024

(54) CONCENTRIC BREATHING CIRCUIT WITH BOOST ZONE

(71) Applicant: Covidien LP, Mansfield, MA (US)

(72) Inventors: Maurizio LISTO, Modena (IT); Stanley B. KAUS, Longmont, CO (US); Stefano BORALI, Finale Emilia (IT); Donald J. NOVKOV, Encinitas, CA (US); Matthew J PHILLIPS, Carlsbad, CA (US)

(73) Assignee: Covidien LP, Mansfield, MA (US)

(21) Appl. No.: 18/356,330

(22) Filed: Jul. 21, 2023

Related U.S. Application Data
(60) Provisional application No. 63/391,513, filed on Jul. 22, 2022.

(51) Int. Cl. A61M 16/08; A61M 16/10; A61M 16/00; A61M 16/16

(52) U.S. Cl. CPC A61M 16/0883; A61M 16/1095; A61M 16/022; A61M 16/16; A61M 2205/3368; A61M 2025/0039

(57) ABSTRACT

Patient breathing circuits with a boost-heated extension. In an example, a patient breathing circuit includes an outer conduit having a first end connectable to a patient interface and a second end connectable to a humidifier; an inner conduit positioned within the first conduit, wherein a first lumen is defined within an interior of the inner conduit and a second lumen is defined between an exterior of the inner conduit and an interior of the outer conduit; a first heater wire coupled to the inner conduit; and a boost-heated expiratory extension having a first end extending from the outer conduit and a second end connectable to an expiratory port of a ventilator, wherein the boost-heated expiratory extension includes at least one of a second heater wire or a metallic post for heating gases flowing through the boost-heated expiratory extension.

### CROSS-REFERENCE TO RELATED APPLICATIONS

This application claims the benefit of U.S. Provisional Application No. 63/391,513 filed 22 Jul. 2022, entitled “Concentric Breathing Circuit with Boost Zone,” which is incorporated herein by reference in its entirety.

### INTRODUCTION

Medical ventilator systems are used to provide ventilatory and supplemental oxygen support to patients. These ventilators typically comprise a connection for pressurized gas (air, oxygen) that is delivered to the patient through a conduit or tubing. The gas that is delivered to the patient may be humidified before the gas reaches the patient. While the humidification provides benefits, such as increasing the comfort of ventilation for the patient, the additional moisture introduced into the system may cause condensation to occur in portions of the breathing circuit. The condensation may produce adverse effects for both the patient and ventilation equipment.

It is with respect to this general technical environment that aspects of the present technology disclosed herein have been contemplated. Furthermore, although a general environment is discussed, it should be understood that the examples described herein should not be limited to the general environment identified herein.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Among other things, aspects of the present disclosure include ventilation systems with a boost zone for heating exhaled gases and methods for controlling such systems. In an aspect, the technology relates to a patient breathing circuit that includes an outer conduit having a first end connectable to a patient interface and a second end connectable to a humidifier; an inner conduit positioned within the first conduit, wherein a first lumen is defined within an interior of the inner conduit and a second lumen is defined between an exterior of the inner conduit and an interior of the outer conduit; a first heater wire coupled to the inner conduit; and a boost-heated expiratory extension having a first end extending from the outer conduit and a second end connectable to an expiratory port of a ventilator, wherein the boost-heated expiratory extension includes at least one of a second heater wire or a metallic post for heating gases flowing through the boost-heated expiratory extension.

In an example, the first lumen is configured to carry humidified breathing gases from the humidifier to the patient interface; and the second lumen is configured to carry exhaled gases from the patient interface to the boost-heated expiratory extension. In another example, the breathing circuit further includes a third heater wire coupled to the outer conduit. In yet another example, the boost-heated expiratory extension includes the metallic post. In still another extension, the boost-heated expiratory extension includes the second heater wire. In still yet another example, the outer conduit has a first length, and the boost-heated expiratory extension has a second length, wherein the second length is less than 50% of the first length.

In another aspect, the technology relates to a patient breathing circuit that includes an expiratory conduit for carrying expiratory gases from a patient interface towards an expiratory port of a ventilator; a metallic post connectable to the expiratory conduit for carrying expiratory gases from the expiratory conduit towards to the expiratory port; and a heater coupled to the metallic post to heat the metallic post.

In an example, the metallic post has a length that is less than or equal to 6 inches. In another example, the heater is configured to heat the metallic post to a temperature greater than 50 degrees Celsius. In yet another example, the breathing circuit further includes an insulating cover covering the metallic post.

In another aspect, the technology relates to a method, performed by a humidifier, for controlling heating within a concentric patient breathing circuit including an inspiratory conduit concentrically positioned within an expiratory conduit. The method includes receiving a first temperature measurement of breathing gases flowing through the inspiratory conduit of the breathing circuit coupled to a patient interface; based on the first temperature measurement, generating a first heater wire control signal for a first heater wire coupled to the inspiratory conduit, the first heater wire control signal configured to cause breathing gases entering the patient interface to reach a patient-specific temperature; receiving a second temperature measurement for exhaled gases flowing through a boost-heated expiratory extension, the boost-heated expiratory extension extending from the expiratory conduit to an expiratory port of a ventilator; and generating a boost control signal for controlling at least one of a heater wire or a heater of the boost-heated expiratory extension, the boost control signal configured to heat exhaled gases entering the expiratory port to a heat-boosted target temperature, the heat-boosted target temperature being at least 5 degrees Celsius greater than the patient-specific temperature.

In an example, the method further includes generating a second heater wire control signal for a second heater wire coupled to the expiratory conduit. In another example, the second heater wire control signal is based on the first temperature measurement. In yet another example, the first heater wire control signal is a pulse-width-modified (PWM) signal. In still another example, the heat-boosted target temperature is at least 45 degrees Celsius and less than 60 degrees Celsius.

It is to be understood that both the foregoing general description and the following Detailed Description are explanatory and are intended to provide further aspects and examples of the disclosure as claimed.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 depicts an example ventilation system with a concentric breathing circuit with a boost-heated expiratory extension.

FIG. 2 depicts another example ventilation system with a concentric breathing circuit with a boost-heated expiratory extension.

FIG. 3 depicts another example ventilation system with a concentric breathing circuit with a boost-heated expiratory extension.

FIG. 4 depicts another example ventilation system with a concentric breathing circuit with a boost-heated expiratory extension.

FIG. 5 depicts another example ventilation system with a concentric breathing circuit with a boost-heated expiratory extension.

FIG. 6 depicts a perspective view of another example ventilation system with a concentric breathing circuit with a boost-heated expiratory extension.

FIG. 7 is a diagram illustrating an example of a medical ventilator connected to a human patient.

FIG. 8 depicts an example diagram of a breathing circuit with multiple heated zones available with the present technology.

FIG. 9 depicts an example method for controlling a breathing circuit having a boost zone.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

As discussed briefly above, medical ventilators are used to provide breathing gases to patients who are otherwise unable to breathe sufficiently, and those breathing gases may be humidified by a humidifier. The process of humidifying the breathing gases necessarily introduces moisture into the breathing gases and into the breathing circuit that carries the breathing gases. In some cases, the additional moisture from the humidification causes condensation (e.g., water droplets) to form in the interior of the conduits forming the breathing circuit. This condensation is sometimes referred to as “rainout.” Rainout can produce undesirable effects for both the patient and for the ventilation system providing the breathing gases. For instance, in some cases, the condensed moisture may fill or partially fill a conduit of the breathing circuit, which causes an occlusion. As another example, the condensed water may interfere with the ventilation system, such as with the expiratory sensors and/or filters. Condensed water droplets may coat or saturate the sensors and cause inaccurate measurements, and the water may also saturate the expiratory filter, which degrades the performance of the filter.

When a rainout event occurs that interferes with ventilation and/or has the potential to have a negative effect on the patient, the water must be emptied from the breathing circuit and/or the breathing circuit must be changed. Disconnection of the breathing circuit while the patient is undergoing ventilation also has its own negative effects. Such disconnection may result in decruitment or atelectasis of the lung. In addition, disconnecting or opening the breathing circuit may expose the air in the room to any pathogens that are in the breathing circuit. Moreover, the condensation itself may increase the likelihood and/or rate of growth of such pathogens.

The condensation or rainout forms where the temperature of the humidified breathing gases drops below its dew point. Accordingly, heating the breathing gases to prevent such drops in temperature is one way to prevent rainout. Heating of the breathing gases, however, has several limitations. For instance, there is a desired temperature for the breathing gases to be delivered to the patient (e.g., a patient-specific temperature). In simple terms, delivering gases that are too hot may cause discomfort or injury to the patient. Similarly, increasing the temperature of conduits or portions of the breathing circuit that are close to the patient's face or in positions within the patient's reach may create potential dangers for the patient. In addition, heating the breathing gases near a patient, even exhaled gases, may create a risk of rebreathing those gases or having heat transfer to inhaled breathing gases.

The present technology allows for heating breathing gases in manner that substantially reduces the likelihood of rainout while still protecting the patient and breathing gases from the negative effects of heating discussed above. During ventilation with a breathing circuit having a heated inspiratory limb, breathing gases are most likely to rainout in the expiratory limb at positions that are further from the patient. For instance, as the exhaled gases travel from the patient back to the ventilator, the exhaled gases lose heat. If enough heat is lost for the breathing gases to drop below their dew point, rainout may occur.

The present technology provides a boost-heated expiratory extension in the breathing circuit that creates a boost zone of extra heat into the exhaled gases at a position that is closer to the ventilator than the patient. One example of such a breathing circuit includes a concentric segment that forms at least part of the inspiratory limb and the expiratory limb of the breathing circuit. A final segment between the concentric segment and the ventilator then includes the boost-heated expiratory extension to heat the exhaled gases prior to the exhaled gases entering the ventilator.

By incorporating the boost-heated expiratory extension in such a manner, the breathing gases can be heated to temperatures that would be unsuitable for breathing because the possibility of such gases being breathed by the patient are incredibly low. In addition, heating of the boost-heated expiratory extension at a position that is closer to the ventilator also reduces the likelihood that the patient will come into physical contact with the boost-heated expiratory extension. Additional benefits and advantages are also provided by the present technology as discussed herein.

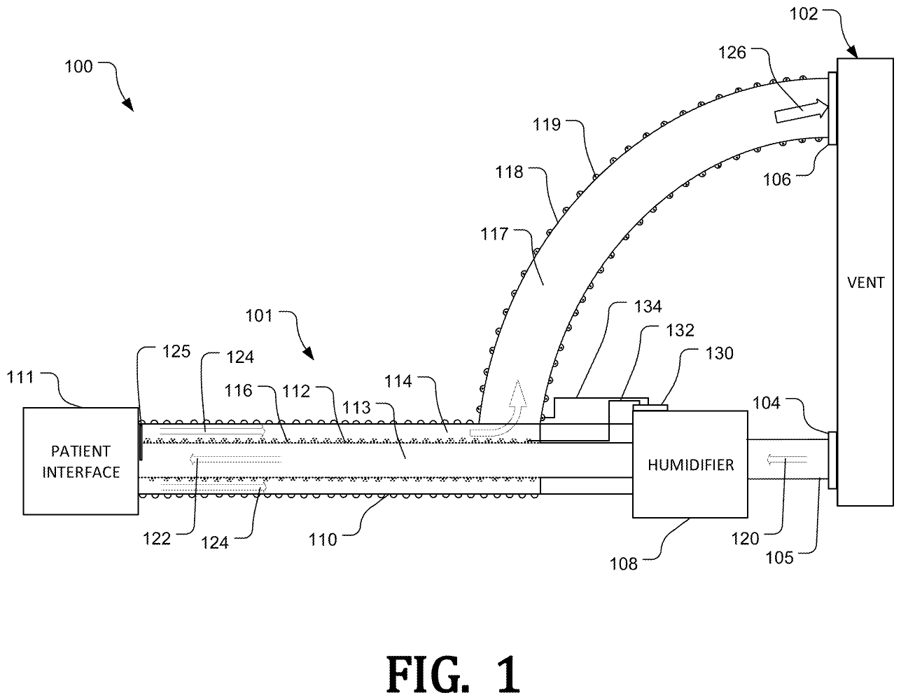

FIG. 1 depicts an example ventilation system 100 with a concentric breathing circuit 101 with a boost-heated expiratory extension 118. The ventilation system 100 includes a ventilator 102, a humidifier 108, and a patient interface 111. The ventilator 102 generates a positive pressure of dry breathing gases 120 that are delivered from an inspiratory port 104 of the ventilator 102 through an initial conduit 105 extending from the inspiratory port 104 to the humidifier 108. The dry breathing gases 120 are then received by a humidifier 108 that humidifies the dry breathing gases 120 to form humidified breathing gases 122.

The humidified breathing gases 122 flow through an inner lumen 113 of an inner conduit 112 of the concentric breathing circuit 101. The concentric breathing circuit 101 is connected to a patient interface 111 that provides the humidified breathing gases 122 to the patient. The patient interface 111 may be in invasive or a non-invasive patient interface 111. For instance, the patient interface 111 may be an endotracheal tube, mask, or other type of patient interface 111. In some examples, the patient interface 111 may also be swappable from one form to another. For instance, a non-invasive patient interface 111 may be removed from the outer conduit 110, and an invasive patient interface 111 may be connected (or vice versa).

When the patient exhales, the exhaled gases 124 are carried by an outer lumen 114 that is formed between an outer conduit 110 and the inner conduit 112 of the concentric breathing circuit 101. For instance, the concentric breathing circuit 101 includes an outer conduit 110 and an inner conduit 112 located within the outer conduit 110. The inner conduit 112 includes the inner lumen 113, and the outer lumen 114 is formed between the outer wall of the inner conduit 112 and the inner wall of the outer conduit 110. The inner lumen 113 carries the humidified breathing gases 122 to the patient interface 111, and the outer lumen 114 carries the exhaled gases away from the patient interface 111. In some examples, the outer conduit 110 may be referred to as the expiratory conduit 110 and the inner conduit 112 may be referred to as the inspiratory conduit 112.

A boost-heated expiratory extension 118 extends from a portion of the concentric breathing circuit 101 that carries the gases 124 to an expiratory port 106 of the ventilator 102. The boost-heated expiratory extension 118 may extend from an end of the concentric breathing circuit 101 that is closer to the humidifier 108 than the patient interface 111. For instance, the boost-heated expiratory extension 118 may begin near the connection point of the concentric breathing circuit 101 and the humidifier 108. In some examples, the boost-heated expiratory extension 118 may extend from a point on the concentric breathing circuit 101 that is within 10 inches of the connection point of the concentric breathing circuit 101 to the humidifier 108. In other examples, the boost-heated expiratory extension 118 may connect to, and extend from, the humidifier 108, and the exhaled gases 124 in the outer lumen 114 pass through the humidifier 108 before entering the boost-heated expiratory extension 118.

The outer conduit 110 may include a port or through hole that allows the gases 124 to flow from the outer lumen 114 into a heat-boosted lumen 117 formed by the boost-heated expiratory extension 118. For instance, the boost-heated expiratory extension 118 may be formed by another conduit that extends from the outer conduit 110. In some examples, the boost-heated expiratory extension 118 may be integrally formed with the concentric breathing circuit 101 (e.g., non-removable). In other examples, the boost-heated expiratory extension 118 may be removable from the concentric breathing circuit 101. For instance, different boost-heated expiratory extensions 118 may be available that have different lengths, heating capabilities, and/or other characteristics.

The conduit forming the boost-heated expiratory extension 118 is heated, and one or more of the conduits forming the concentric breathing circuit 101 may also be heated. In the example depicted, the boost-heated expiratory extension 118 is heated by a boost heater wire 119 that is coiled or wrapped around the conduit forming the boost-heated expiratory extension 118. In some examples, the heater wire 119 may be a high-resistance alloy wire, such as a Nickel-Chromium wire. When an electrical current is run through the boost heater wire 119, the boost heater wire 119 generates heat. The generated heat transfers thermal energy to the exhaled gases 124 that enter the boost-heated expiratory extension 118. Thus, by the time the exhaled gases 124 reach the expiratory port 106, the exhaled gases 124 have been heated to form heat-boosted gases 126.

Heating the exhaled gases 124 to form the heat-boosted gases 126 reduces the likelihood of rainout occurring within the boost-heated expiratory extension 118 or within the ventilator 102 after the heat-boosted gases 126 enter the expiratory port 106. As a result, the longevity and reliability of the sensors, filters, or other components of the ventilator 102 at the expiratory port 106 is increased. As discussed above, when rainout occurs in the expiratory limb and/or within the expiratory port 106 of the ventilator 102, the resultant condensed liquid may interfere with components of the ventilator 102. For instance, the expiratory filter may become saturated with the liquid and no longer function properly. Similarly, the liquid may coat sensors and/or valves in the expiratory port 106 in a manner that adversely affects their performance. When the breathing gases 124 are heated to become form heat-boosted gases 126, the likelihood of rainout or condensation is reduced or eliminated. As such, when the form heat-boosted gases 126 flow into the ventilator 102, liquid does not condense from the heat-boosted gases 126, and the problems associated with liquids interfering with the components of the ventilator 102 can be substantially avoided or alleviated.

One or more of the outer conduit 110 or the inner conduit 112 may also be heated. In the example depicted in FIG. 1, the inner conduit 112 is heated with an inner-conduit heater wire 116 that is coupled to (e.g., wrapped or coiled around) the inner conduit 112, but the outer conduit 110 does not include a dedicated heater wire. The inner-conduit heater wire 116 may generate heat in a similar manner as the boost heater wire 119. For instance, when an electrical current is run through the inner-conduit heater wire 116, heat is generated from the inner-conduit heater wire 116. The heat that is generated from the inner-conduit heater wire 116 is transferred to both the humidified breathing gases 122 in the inner lumen 113 and the exhaled gases 124 in the outer lumen 114.

Control or modulation of the electrical current delivered through the inner-conduit heater wire 116 controls the temperature of the humidified breathing gases 122 that are ultimately delivered to the patient. With the design of the concentric breathing circuit 101, that same heat also warms the exhaled gases 124, which in turn helps prevent rainout within the outer lumen 114. While rainout is less likely to occur near the patient, the additional heat added to the outer lumen 114 still helps prevent rainout near the patient, which may still be likely in colder environments where the exhaled gases 124 are more likely to drop in temperature over a shorter period.

The inner-conduit heater wire 116 and the boost heater wire 119 may be controlled independently from one another. For instance, a first power signal may be provided to the inner-conduit heater wire 116, and a second power signal may be provided to the boost heater wire 119 to generate two different types or amounts of heat generation. The separate control of the inner-conduit heater wire 116 and the boost heater wire 119 is useful because the temperature of the humidified breathing gases 122 should be specific to needs of the patient, whereas the temperature of the heat-boosted gases 126 may be raised to heat-boosted target temperature irrespective of the patient. For instance, in some examples, the temperature of the humidified breathing gases 122 may be desired to be near the internal temperature of the patient, such as 37 degrees Celsius. The desired or set temperature of breathing gases 122 to be delivered to the patient may be referred to as the patient-specific temperature.

The temperature of the heat-boosted gases 126, however, may be raised to temperatures that are above that patient-specific temperature because the patient is not breathing the heat-boosted gases 126. By raising the heat-boosted gases 126 above the patient-specific temperature, the likelihood of rainout of the heat-boosted gases 126 can be significantly reduced without creating negative effects for the patient. In some examples, the temperature of the heat-boosted gases 126 may be at least 40 degrees Celsius, at least 45 degrees Celsius, or at least 50 degrees Celsius. As another example, the heat-boosted gases 126 may be at least 5 degrees Celsius higher than that of the breathing gases 124 that are delivered to the patient. The temperature of the heat-boosted gases 126 may be measured at the expiratory port 106 of the ventilator 102.

While the temperatures of the heat-boosted gases 126 may be raised to high temperatures, there may be drawbacks to heating the heat-boosted gases 126 too high. For instance, at high temperatures, such as above 60 degrees Celsius, the plastic or material forming the conduit may begin to melt. The heat may also interfere with one or more of the sensors in the expiratory port 106 of the ventilator 102. For example, the expiratory port 106 may include a flow sensor that is a hot-wire-anode-based sensor. Such sensors rely on temperature measurements to measure flow. If the temperature of the heat-boosted gases 126 is too high, the flow sensor may provide inaccurate readings and may cause the ventilator to alarm. Thus, in some examples, the temperature of the heat-boosted gases 126 does not exceed 55 degrees Celsius or 60 degrees Celsius.

Control of the inner-conduit heater wire 116 and the boost heater wire 119 may be provided by the humidifier 108, as shown in the example depicted. For example, the inner-conduit heater wire 116 includes a control wire 132 that connects to an electrical port 130 of the humidifier 108. The boost heater wire 119 also includes a control wire 134 that connects to the electrical port 130 of the humidifier 108. The electrical port 130 may include multiple connection ports or other forms of electrical connections for creating an electrical connection between the humidifier 108 and the control wires 132, 134. Each of the control wires 132, 134 may consist of at least two wires that allows for an electrical current to flow through the control wires 132, 134 and the respective inner-conduit heater wire 116 or boost heater wire 119.

The humidifier 108 may control the heat that is generated from the inner-conduit heater wire 116 and the boost heater wire 119 by providing different electrical signals to the inner-conduit heater wire 116 and the boost heater wire 119. The electrical signals may differ in voltage or other characteristics. In an example, electrical signal includes a pulse-width-modified (PWM) signal that includes a series of voltage pulses that are delivered to the respective heater wire 116, 119. The PWM signal may be characterized by its voltage (e.g., amplitude), frequency, and duty cycle. The voltage and frequency of the electrical signals may remain the same across the electrical signals that transmitted through the inner-conduit heater wire 116 and the boost heater wire 119. The duty cycle, however, may be changed to alter the total power that is transmitted through the inner-conduit heater wire 116 and the boost heater wire 119. A greater duty cycle delivers a greater amount of power, whereas a lower duty cycle delivers a smaller amount of power. Of note, while the control of the inner-conduit heater wire 116 and the boost heater wire 119 are discussed in this example as being controlled by the humidifier 108, in other examples, control of the inner-conduit heater wire 116 and/or the boost heater wire 119 may be provided by the ventilator 102 or another stand-alone device.

A temperature probe 125 may also be incorporated into the concentric breathing circuit 101. The temperature probe 125 may be positioned at an end of the concentric breathing circuit 101 that is closest to the patient interface 111. The temperature probe 125 is positioned to detect or measure the temperature of the humidified breathing gases 122 flowing through the inner lumen 113 of the inner conduit 112. In some examples, the temperature probe 125 also includes a humidity sensor that senses the humidity of the humidified breathing gases 122. The temperature probe 125 may be integrated into a connector that connects the concentric breathing circuit 101 to the patient interface 111. Additional temperature sensors may also be included in other positions throughout the concentric breathing circuit 101 and/or the boost-heated expiratory extension 118.

The measurements from the temperature probe 125 are transmitted to the humidifier 108, where the humidifier 108 may adjust its operations based on the received measurements. For instance, the humidifier 108 may adjust the temperature and/or humidity settings of the humidifier 108 itself (e.g., heaters within the humidifier 108), and/or the humidifier 108 may adjust the controls of the inner-conduit heater wire 116 and/or boost heater wire 119. As an example, the humidifier 108 may adjust the electrical signal transmitted through the inner-conduit heater wire 116 to cause the temperature of the humidified breathing gases 122 to be at the patient-specific temperature (e.g., 37 degrees Celsius) when the humidified breathing gases 122 reach the temperature probe 125.

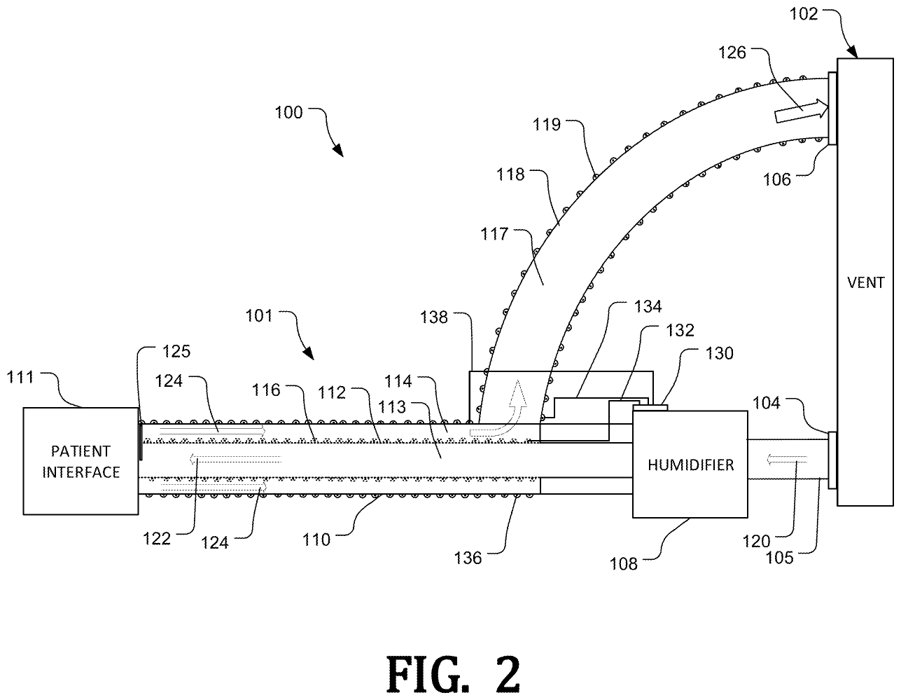

FIG. 2 depicts another example of the ventilation system 100 with the concentric breathing circuit 101 with the boost-heated expiratory extension 118. The ventilation system 100 depicted in FIG. 2 is substantially the same as the ventilation system 100 depicted in FIG. 1 with the exception that the outer conduit 110 also include as an outer-conduit heater wire 136. The outer-conduit heater wire 136 operates in a similar manner as the inner-conduit heater wire 116 and the boost heater wire 119. For instance, when an electrical current runs through the outer-conduit heater wire 136, heat is generated by the outer-conduit heater wire 136.

The heat that is generated from the outer-conduit heater wire 136 heats the exhaled gases 124 and the humidified breathing gases 122. However, the heating effect from the outer-conduit heater wire 136 may affect the temperature of the exhaled gases 124 more directly than the humidified breathing gases 122. The use the outer-conduit heater wire 136 may reduce the likelihood of rainout of the exhaled gases 124 while in the outer lumen 114.

The humidifier 108 may also control the electrical signal that is transmitted through the outer-conduit heater wire 136. For example, a control wire 138 may extend from the outer-conduit heater wire 136 to the electrical port 130 of the humidifier 108. The humidifier 108 may control the inner-conduit heater wire 116 and the outer-conduit heater wire 136 together to ensure that the humidified breathing gases 122 are at the patient-specific temperature when the humidified breathing gases 122 reach the patient interface 111. For example, the electrical signals that are transmitted to the inner-conduit heater wire 116 and the outer-conduit heater wire 136 may both be based on the measurement(s) from the temperature probe 125.

The ability to separately control the inner-conduit heater wire 116, boost heater wire 119, and the outer-conduit heater wire 136 provides three configurable heating zones. The first heating zone is that of the inner lumen 113, the second heating zone is that of the outer lumen 114, and the third heating zone is that of the heat-boosted lumen 117. By using the three heating zones, the temperatures of the respective gases may be heated in manners that are most appropriate for the patient and also preventing rainout with the conduits.

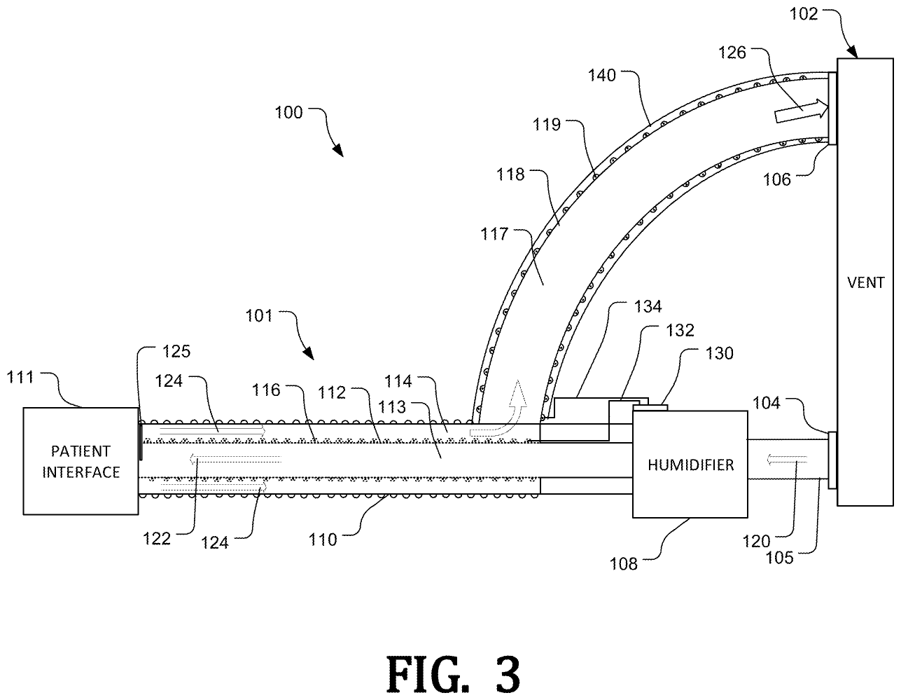

FIG. 3 depicts another example ventilation system with a concentric breathing circuit with a boost-heated expiratory extension. The ventilation system 100 depicted in FIG. 3 is substantially the same as the ventilation system 100 depicted in FIG. 1 with the exception that the boost-heated expiratory extension 118 is covered by an insulator or jacket 140. The jacket 140 covers at least a portion of the conduit forming the boost-heated expiratory extension 118.

The jacket 140 insulates the boost-heated expiratory extension 118 to help retain heat within the jacket 140. For instance, the jacket 140 helps retain heat generated from the boost heater wire 119. Even without the boost heater wire 119, the jacket 140 still retains additional heat within the exhaled gases 124 that flows through the exhaled gases 124.

The jacket 140 may be integrated with the boost-heated expiratory extension 118 (e.g., not removable from the conduit). In other examples, the jacket 140 may be separable or removable from the conduit forming the boost-heated expiratory extension 118. In such examples, the jacket 140 may be reusable. In some examples, the jacket 140 may also be partially filled with an insulating material to provide additional heat insulation.

In addition to retaining heat within the boost-heated expiratory extension 118, the jacket 140 may provide protection to clinicians or users of the ventilation system 100. In some examples, the boost heater wire 119 may be heated to levels that are uncomfortable to human touch over a period of time. The jacket 140 may prevent at least a portion of the heat from the boost heater wire 119 from reaching the exterior surface of the jacket 140 where a clinician or user may grab the boost-heated expiratory extension 118.

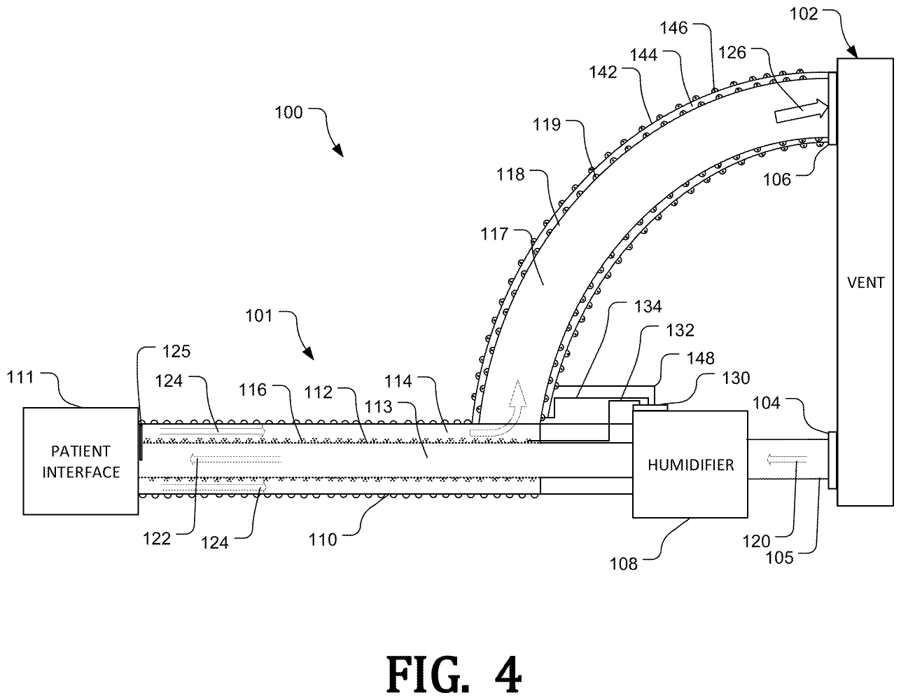

FIG. 4 depicts another example ventilation system 100 with the concentric breathing circuit 101 with the boost-heated expiratory extension 118. The ventilation system 100 depicted in FIG. 4 is substantially similar to the ventilation system 100 depicted in FIG. 1 with the exception that the boost-heated expiratory extension 118 is formed from two concentric conduits, including an addition boost-heated outer conduit 142. The boost-heated outer conduit 142 may include a boost-heated outer-conduit heater wire 146. The boost-heated outer-conduit heater wire 146 may be controlled by the humidifier 108 via a control wire 148 extending from the boost-heated outer-conduit heater wire 146 to the electrical port 130 of the humidifier 108.

With the boost-heated outer conduit 142, a boost-heated outer gap or lumen 144 is formed between the boost-heated outer conduit 142 and the boost-heated inner conduit. The boost-heated outer lumen 144, however, may not have any breathing or exhaled breathing gases flowing through the boost-heated outer lumen 144. Rather, the boost-heated outer conduit 142 and the boost-heated outer lumen 144 serve to heat and insulate the exhaled gases 124 flowing through the boost-heated expiratory extension 118 to form the heat-boosted gases 126. As should be appreciated, providing additional heat from the boost-heated outer-conduit heater wire 146 provides additional heat energy to the exhaled gases 124 flowing through the boost-heated expiratory extension 118.

By including the boost-heated outer conduit 142 with the boost-heated outer-conduit heater wire 146, the length of the boost-heated expiratory extension 118 may be made shorter as the heat transfer from both the boost heater wire 119 and the boost-heated outer-conduit heater wire 146 are able to heat the exhaled gases 124 more quickly. The length of the boost-heated expiratory extension 118 may be dependent on how much thermal energy can be transferred to the exhaled gases 124. For instance, where high amounts of thermal energy may be transferred to the exhaled gases 124, the length of the boost-heated expiratory extension 118 may be shorter. In contrast, where lower amounts of thermal energy may be transferred to the exhaled gases 124, the length of the boost-heated expiratory extension 118 may need to be greater to reach desired temperatures for the heat-boosted gases 126.

The length of the boost-heated expiratory extension 118 may be substantially shorter than the length of the concentric breathing circuit 101. In some examples, the length of the boost-heated expiratory extension 118 may less than 50% of the length of the concentric breathing circuit 101. In other examples, the length of the boost-heated expiratory extension 118 may be less than 25% or 10% of the length of the concentric breathing circuit 101.

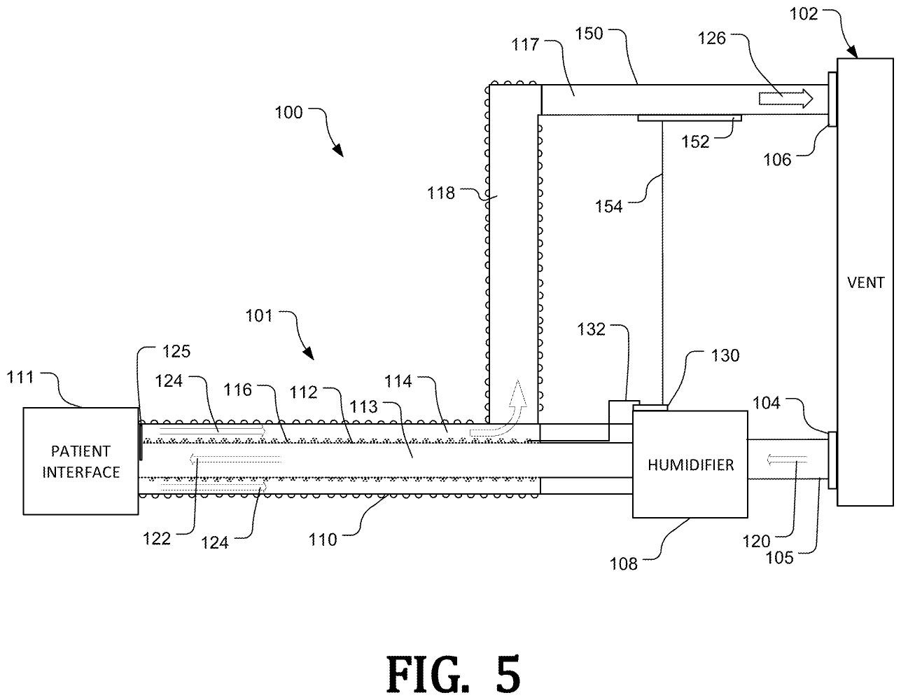

FIG. 5 depicts another example ventilation system 100 with the concentric breathing circuit 101 with the boost-heated expiratory extension 118. The ventilation system 100 depicted in FIG. is substantially similar to the ventilation system 100 depicted in FIG. 1 with the exception of the configuration of the boost-heated expiratory extension 118.

In the example depicted in FIG. 5, the boost-heated expiratory extension 118 includes a first portion made from a conduit made of a plastic or other non-metallic material, similar to the examples above, and a second metallic tube or post 150 that includes the heat-boosted lumen 117. The metallic post 150 may be made from metal material that has a high heat conductivity to transfer heat energy to the breathing gases 124 flowing into the metallic post 150. Because the metallic post 150 is a metal tube with increased surface area as compared to the coiled wire around a conduit, such as the respective heater wire 119 discussed above, the heat transfer to the breathing gases 124 is increased. As a result, the metallic post 150 may be made fairly short and still achieve quick rises in temperature of the breathing gases 124 to form the heat-boosted gases 126. In some examples, the length of the metallic post 150 may be less than 12 inches, 6 inches, or 3 inches. The metallic post 150 may need to be heated to higher temperatures as the length of the metallic post 150 decreases.

A heater 152 is coupled to the metallic post 150 to heat the metallic post 150. The heater 152 may be controlled by the humidifier 108. In the example depicted, a control wire 154 extends from the heater 152 to the electrical port 130 of the humidifier 108. The humidifier 108 may then send electrical signals to the heater 152 to cause the heater 152 to heat the metallic post 150 to a desired temperature. In some examples, the metallic post 150 may include a temperature sensor that senses the temperature of the inner surface of the metallic post 150 and/or the form heat-boosted gases 126. Accordingly, the metallic post 150 may be heated based on temperature measurements of the metallic post 150 and/or the heat-boosted gases 126. In some examples, the metallic post 150 may be heated to temperatures in excess of 50 or 60 degrees Celsius. For instance, the metallic post 150 may be heated to 60-75 degrees Celsius.

In some examples, the metallic post 150 and heater 152 may be standalone heat-boost device that connects to the expiratory port 106 of the ventilator 102 and provides a connection to an expiratory limb of a breathing circuit. The temperature of the metallic post 150 may be a present temperature and/or may be user-adjustable. Thus, the standalone heat-boost device may be incorporated into many different types of ventilation systems and breathing circuits.

Due to the higher temperatures of the metallic post 150, the metallic post 150 may be wrapped with an insulating layer. The exterior of the insulating layer may remain cool enough to touch or handle when the metallic post 150 is heated. In addition, the connectors that connect the metallic post 150 to the plastic conduit portion of the boost-heated expiratory extension 118 and the expiratory port 106 may be constructed of a material or in a manner that prevents the heat of the metallic post 150 from transferring to the other components. For instance, the connectors for the metallic post 150 may be made of silicone or another similar material that is heat resistant.

In the example depicted, the plastic conduit portion of the boost-heated expiratory extension 118 does not include the respective heater wire 119. In other examples, however, the boost-heated expiratory extension 118 may also include the respective heater wire 119 in addition to the metallic post 150 and metallic post 150.

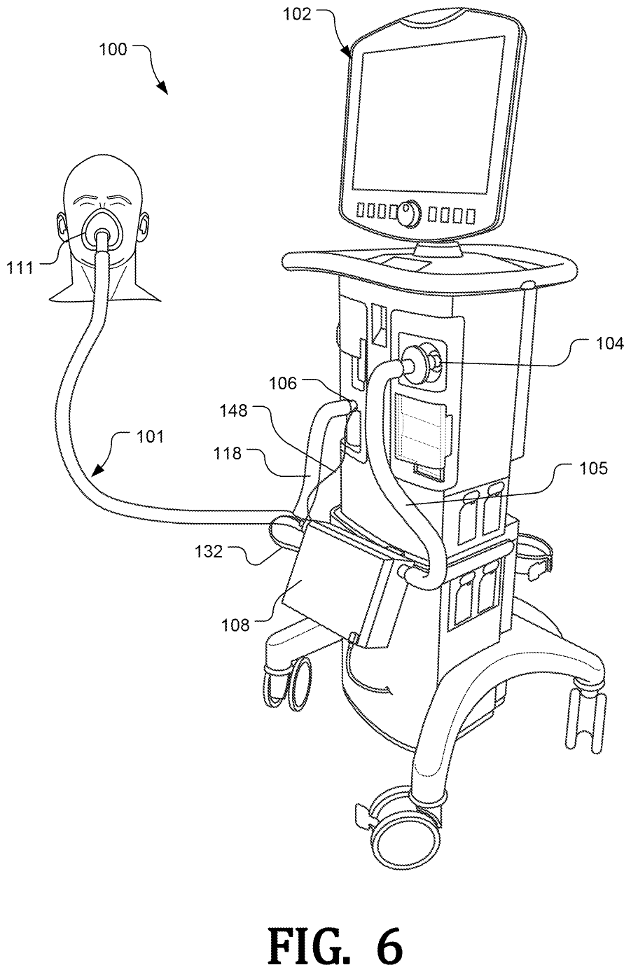

FIG. 6 depicts a perspective view of another example ventilation system 100 with a concentric breathing circuit 101 with a boost-heated expiratory extension 118. In the example ventilation system 100 depicted, an initial conduit 105 extends from an inspiratory port 104 of a ventilator 102. The initial conduit 105 connects in an input port of a humidifier 108 which humidifies the breathing gases. The concentric breathing circuit 101 extends from the output port of the humidifier 108 to a patient interface 111. The boost-heated expiratory extension 118 extends from the concentric breathing circuit 101 to an expiratory port 106 of the ventilator 102.

The concentric breathing circuit 101 and/or boost-heated expiratory extension 118 may have any of the configurations discussed above. In the example depicted in FIG. 6, a control wire 132 for controlling at least one heater wire of the concentric breathing circuit 101 is shown as being connected to the concentric breathing circuit 101 and the humidifier 108. In addition, a control wire 148 for controlling a heater wire and/or heater of the boost-heated expiratory extension 118 is shown as being connected to the humidifier and the boost-heated expiratory extension 118.

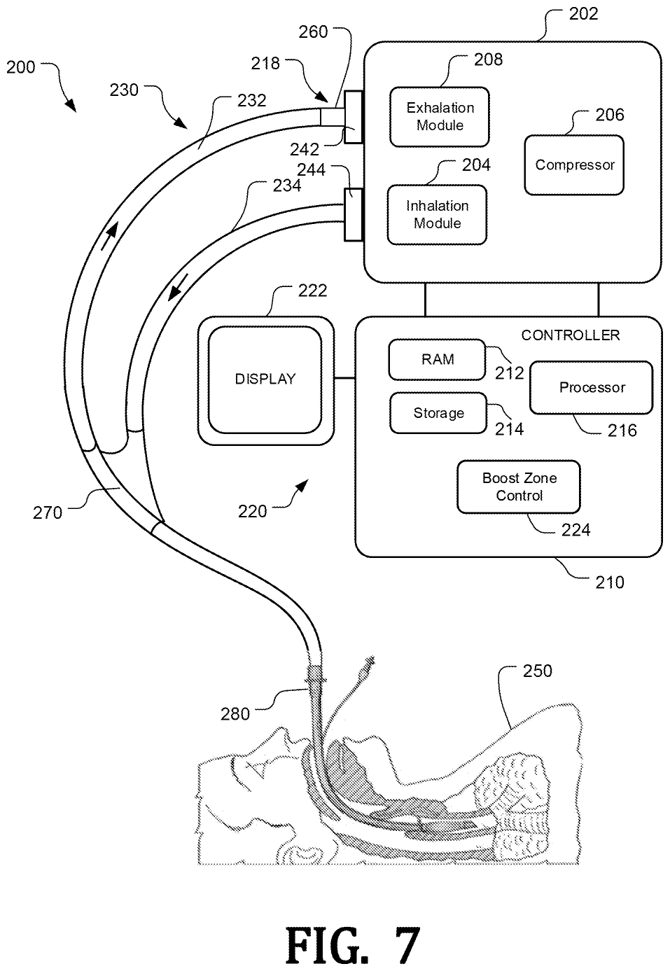

FIG. 7 is a diagram illustrating an example of a medical ventilation system 200 connected to a human patient 250. The ventilation system 200 may provide positive pressure ventilation to the patient 250. Ventilation system 200 includes a pneumatic system 202 (also referred to as a pressure generating system 202) for circulating breathing gases to and from patient 250 via the ventilation tubing system 230, which couples the patient to the pneumatic system via an invasive (e.g., endotracheal tube, as shown) or a non-invasive (e.g., nasal mask) patient interface.

Ventilation tubing system 230 may be a two-limb (shown) for carrying gases to and from the patient 250. In a two-limb example, a fitting, typically referred to as a “wye-fitting” 270, may be provided to couple a patient interface 280 to an inhalation limb 234 and an exhalation limb 232 of the ventilation tubing system 230. The inhalation limb 234 extends from an inspiratory port 244 and the exhalation limb extends from an expiratory port 242.

A boost-heated expiratory extension 218 in the form of a metallic post 260 is included at the end of the expiratory limb 232. The boost-heated expiratory extension 218 extends between the conduit of the expiratory limb 232 and the expiratory port 242. The metallic post 260 may be substantially the same as the metallic post 150 discussed above. A heater may also be included in the system to heat the metallic post. While a humidifier is not explicitly depicted in FIG. 7, a humidifier may be provided to humidify the breathings gases. In examples where the humidifier is included, the humidifier may control the heater to heat the metallic post 260. In the example depicted, however, the heater of the metallic post 260 is controlled by the ventilator.

Pneumatic system 202 may have a variety of configurations. In the present example, system 202 includes an exhalation module 208 coupled with the exhalation limb 232 and an inhalation module 204 coupled with the inhalation limb 234. Compressor 206 or other source(s) of pressurized gases (e.g., air, oxygen, and/or helium) is coupled with inhalation module 204 to provide a gas source for ventilatory support via inhalation limb 234. The pneumatic system 202 may include a variety of other components, including mixing modules, valves, sensors, tubing, accumulators, filters, etc., which may be internal or external sensors to the ventilator (and may be communicatively coupled, or capable communicating, with the ventilator).

Controller 210 is operatively coupled with pneumatic system 202, signal measurement and acquisition systems, and an operator interface 220 that may enable an operator to interact with the ventilation system 200 (e.g., change ventilation settings, select operational modes, view monitored parameters, etc.). Controller 210 may include memory 212, one or more processors 216, storage 214, and/or other components of the type found in command and control computing devices. The controller 210 may also include a boost-zone control module or algorithm 224 that controls the heating of the boost-heated expiratory extension 218, such as by controlling a heater that heats the metallic post 260. In the depicted example, operator interface 220 includes a display 222 that may be touch-sensitive and/or voice-activated, enabling the display 222 to serve both as an input and output device.

The memory 212 includes non-transitory, computer-readable storage media that stores software that is executed by the processor 216 and which controls the operation of the ventilation system 200. For instance, the memory may store instructions that when executed by the processor 216 perform the operations described herein. In an example, the memory 212 includes one or more solid-state storage devices such as flash memory chips. In an alternative example, the memory 212 may be mass storage connected to the processor 216 through a mass storage controller (not shown) and a communications bus (not shown). Although the description of computer-readable media contained herein refers to a solid-state storage, the computer-readable storage media may be any available media that can be accessed by the processor 216. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between components of the ventilator system or between the ventilator system and other therapeutic equipment and/or remote monitoring systems may be conducted over a distributed network, as described further herein, via wired or wireless means. Further, the present methods may be configured as a presentation layer built over the TCP/IP protocol. TCP/IP stands for “Transmission Control Protocol/Internet Protocol” and provides a basic communication language for many local networks (such as intra- or extranets) and is the primary communication language for the Internet. Specifically, TCP/IP is a bi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination.

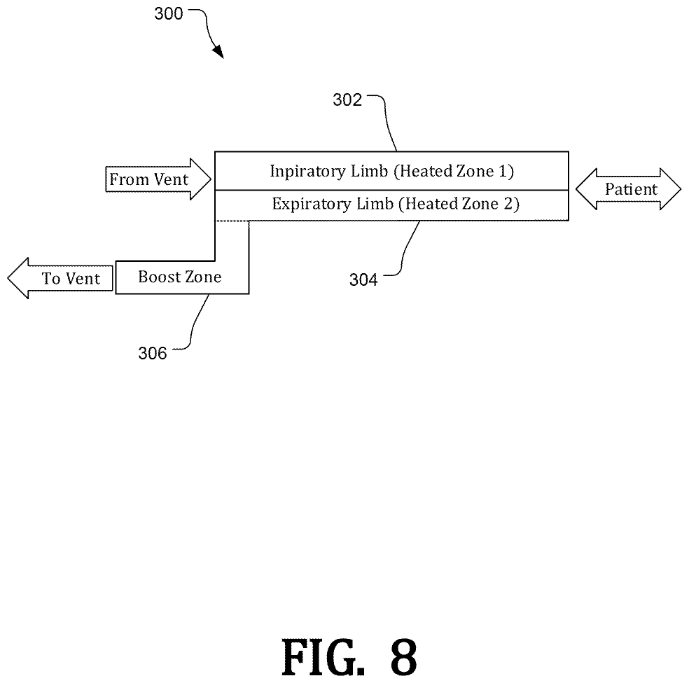

FIG. 8 depicts an example diagram of a breathing circuit 300 with multiple heated zones available with the present technology. The breathing circuit 300 includes a first heated zone 302 that is within the inspiratory limb of the breathing circuit 300. A second heated zone 304 is within the expiratory limb of the breathing circuit. A third heating zone, referred to as a boost zone 306, is within the boost-heated expiratory extension that extends from the expiratory limb to the ventilator. The inspiratory limb and the expiratory limb may be formed as concentric conduits or separated conduits.

The temperature of each of the first heated zone 302, the second heated zone 304, and the boost zone 306 may be separately controlled through different heating elements. For instance, the first heated zone 302 may be controlled via an electrical signal being passed through a heater wire of the inspiratory limb. The second heated zone 304 may be controlled via an electrical signal being passed through a heater wire of the expiratory limb. The boost zone 306 may be controlled by an electrical signal being passed through a heater wire of the boost-heated expiratory extension and/or control of a heater of a metallic post between the expiratory limb and the ventilator.

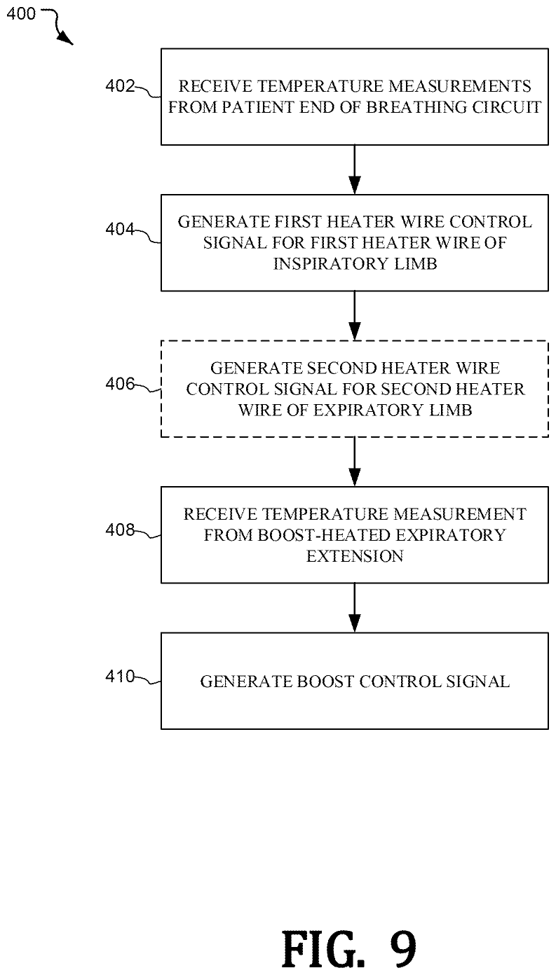

FIG. 9 depicts an example method 400 for controlling a breathing circuit having a boost zone. The operations of method 400 may be performed by one or more devices of the ventilation system. For instance, the operations of method 400 may be performed by a humidifier to control the respective heating elements of the different heater zones of the present technology.

At operation 402, one or more temperature measurements are received from a patient end of a breathing circuit. For instance, the temperature measurement may include a temperature measurement from a temperature sensor or probe at the end of a concentric breathing circuit. Accordingly, the temperature measurement may indicate the temperature of the humidified breathing gases at or near the patient interface.

At operation 404, based on the one or more temperature measurements received in operation 402, a first heater wire control signal for a first heater wire of the inspiratory limb is generated. The first heater wire control signal may be a PWM signal that is intended to increase or decrease the heat produced by the heater wire of the inspiratory limb. For instance, if the temperature measurement indicates the temperature of the breathing gases is below the patient-specific temperature, the first heater wire control signal increases the energy delivered to the first heater wire to increase the temperature the breathing gases delivered to the patient.

At operation 406, a second heater wire control signal is generated for a second heater wire the expiratory limb. The second heater wire of the expiratory limb may be a heater wire in a concentric breathing circuit. The second heater wire control signal may also be a PWM signal that is intended to increase or decrease the heat produced by the heater wire of the expiratory limb. The second heater wire control signal may also be based on the temperature measurement(s) received in operation 402. For example, where the breathing circuit is a concentric breathing circuit, the heat generated from the heater wire of the expiratory limb also affects the temperature of the breathing gases delivered to the patient.

In some examples where the second heater wire is omitted, operation 406 may also be omitted. In still other examples, the first heater wire control signal may be transmitted to both the heater wire of the inspiratory limb and the heater wire of the expiratory limb. In such examples, operation 406 may also be omitted.

At operation 408, one or more temperature measurements for the boost-heated expiratory extension is received. The temperature measurement may be for the heat-boosted gases entering the expiratory port and/or for a portion of the boost-heated expiratory extension. For instance, the temperature measurement may be for the metallic post of the boost-heated expiratory extension.

At operation 410, a boost control signal is generated based on the temperature measurement(s) received at operation 408. The boost control signal may be an electrical signal to control heat generated for a heater wire of the boost-heated expiratory extension. Alternatively or additionally, the boost control signal may be an electrical signal to control a heater of the metallic post. As an example, the temperature measurement received at operation 408 may be for the temperature of the heat-boosted gases, and the boost control signal may be for increasing or decreasing heat to have the heat-boosted gases reach the heat-boosted target temperature to prevent rainout.

A person of skill in the art will understand that the technology described in the context of a medical ventilator for human patients could be adapted for use with other systems such as ventilators for non-human patients or general gas transport systems. Additionally, a person of ordinary skill in the art will understand that the modeled exhalation flow may be implemented in a variety of breathing circuit setups.

Those skilled in the art will recognize that the methods and systems of the present disclosure may be implemented in many manners and as such are not to be limited by the foregoing aspects and examples. In other words, functional elements being performed by a single or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A patient breathing circuit comprising:
an outer conduit having a first end connectable to a patient interface and a second end connectable to a humidifier;
an inner conduit positioned within the first conduit, wherein a first lumen is defined within an interior of the inner conduit and a second lumen is defined between an exterior of the inner conduit and an interior of the outer conduit;
a first heater wire coupled to the inner conduit; and
a boost-heated expiratory extension having a first end extending from the outer conduit and a second end connectable to an expiratory port of a ventilator, wherein the boost-heated expiratory extension includes at least one of a second heater wire or a metallic post for heating gases flowing through the boost-heated expiratory extension.

2. The patient breathing circuit of claim 1, wherein:
the first lumen is configured to carry humidified breathing gases from the humidifier to the patient interface; and
the second lumen is configured to carry exhaled gases from the patient interface to the boost-heated expiratory extension.

3. The patient breathing circuit of claim 1, further comprising a third heater wire coupled to the outer conduit.

4. The patient breathing circuit of claim 1, wherein the boost-heated expiratory extension includes the metallic post.

5. The patient breathing circuit of claim 1, wherein the boost-heated expiratory extension includes the second heater wire.

6. The patient breathing circuit of claim 1, wherein the outer conduit has a first length, and the boost-heated expiratory extension has a second length, wherein the second length is less than 50% of the first length.

7. A patient breathing circuit comprising:
an expiratory conduit for carrying expiratory gases from a patient interface towards an expiratory port of a ventilator;
a metallic post connectable to the expiratory conduit for carrying expiratory gases from the expiratory conduit towards to the expiratory port; and
a heater coupled to the metallic post to heat the metallic post.

8. The patient breathing circuit of claim 7, wherein the metallic post has a length that is less than or equal to 6 inches.

9. The patient breathing circuit of claim 7, wherein the heater is configured to heat the metallic post to a temperature greater than 50 degrees Celsius.

10. The patient breathing circuit of claim 7, further comprising an insulating cover covering the metallic post.

11. A method, performed by a humidifier, for controlling heating within a concentric patient breathing circuit including an inspiratory conduit concentrically positioned within an expiratory conduit, comprising:
receiving a first temperature measurement of breathing gases flowing through the inspiratory conduit of the breathing circuit coupled to a patient interface;
based on the first temperature measurement, generating a first heater wire control signal for a first heater wire coupled to the inspiratory conduit, the first heater wire control signal configured to cause breathing gases entering the patient interface to reach a patient-specific temperature;
receiving a second temperature measurement for exhaled gases flowing through a boost-heated expiratory extension, the boost-heated expiratory extension extending from the expiratory conduit to an expiratory port of a ventilator; and
generating a boost control signal for controlling at least one of a heater wire or a heater of the boost-heated expiratory extension, the boost control signal configured to heat exhaled gases entering the expiratory port to a heat-boosted target temperature, the heat-boosted target temperature being at least 5 degrees Celsius greater than the patient-specific temperature.

12. The method of claim 11, further comprising generating a second heater wire control signal for a second heater wire coupled to the expiratory conduit.

13. The method of claim 12, wherein the second heater wire control signal is based on the first temperature measurement.

14. The method of claim 11, wherein the first heater wire control signal is a pulse-width-modified (PWM) signal.

15. The method of claim 11, wherein the heat-boosted target temperature is at least 45 degrees Celsius and less than 60 degrees Celsius.
